(19) United States
(12) Patent Application Publication
(10) Pub. No.: US 2020/0155729 A1
(43) Pub. Date: May 21, 2020

(54) DISPOSABLE MEDICAL DEVICE FOR MEDICATION OF SKIN LESIONS AND METHOD THEREFORE

(71) Applicant: Diego FURLAN, Segrate (IT)

(72) Inventor: Diego FURLAN, Segrate (IT)

(21) Appl. No.: 16/683,306

(22) Filed: Nov. 14, 2019

(30) Foreign Application Priority Data
Nov. 16, 2018 (IT) 102018000010390

(51) Int. Cl. A61L 31/16; A61L 31/04

(52) U.S. Cl. CPC A61L 31/16; A61L 31/044

(57) ABSTRACT

A disposable medical device for medication of skin lesions is described, which comprises a sealed and sterilized external envelope (1), inside which a chamber or tray (2) is provided, in which a collagen sheet (3) is accommodated, on which collagen sheet (3) a suspension of epithelial or dermal-epithelial cells, consisting of patient's intact skin shreds, subjected to crushing, shredding and homogenization, is adhered.

### BACKGROUND OF THE INVENTION

The present invention relates to a disposable medical device which is usable for medication of patient's skin lesions, such as wounds, burns, bedsores, chronic ulcers, diabetic foot and the like, and to related preparation and use methods.

The healing of large chronic and acute wounds is a challenging task for operators in the sector: it requires several visits and frequent medication changes and often involves expensive therapeutic procedures. The primary objective is to close these wounds as quickly as possible. In a prepared wound bed, with a good granulation tissue and free of infection, the skin graft is the standard procedure to achieve this goal. Unfortunately, its application is often limited by the insufficient availability of autologous skin grafts. An alternative is the grafting of cultured keratinocytes and/or fibroblasts, possibly autologous. However, due to several complications and to the graft preparing process, which takes a long time, normally two to three weeks, to obtain a fragile and thin layer of cells of autologous cultured skin, the initial optimism for the autograft of autologous cells has gradually decreased.

An example of a technique currently used for the treatment of cutaneous wounds is described in the article “Biologicskin substitutes” by Tania J. Phillips published in the Journal of Dermatologic Surgery and Oncology, New York, N.Y., US, vol. 19, 1993, pages 794-800, XP002929718, FIG. 6, and includes the injection of epithelial cells into a sterile envelope containing a biodegradable mesh. The cells attach themselves to the mesh, multiply and begin to secrete collagen, i.e. proteins which help form connective tissues. Gradually, the cells and collagen proteins cover the mesh inside and outside to form a solid tissue. The replacement skin is sutured with the wound and the mesh gradually dissolves.

Another example is described in WO 03/076604 A2 and includes using a biocompatible cross-linked matrix, on which cultured epithelial cells are inoculated and cultured.

Yet another example is described in the article “A Tissue Culture Perfusion Chamber with a Substratum of Reconstituted Collagen” by Thomas M. Harris, 1996, XP055610127, FIG. 1, and includes using a screen disk covered with collagen placed inside a chamber which is then filled with a culture medium.

### SUMMARY OF THE INVENTION

It is the object of the present invention to solve the problems related to the prior art, allowing a rapid and efficient availability of flaps of engineered tissue to be transplanted onto the lesion of the patient.

To achieve this object, a disposable medical device has been devised with the present invention, which can be made available relatively quickly for the patient.

The use of a collagen sheet, forming a perfectly uniform flat surface which is free from perforations, reliefs and/or depressions, is particularly suitable for the uniform growth of an epithelial replacement layer to be applied in an effectively curative manner and in a short time to the patient's wound.

This disposable device, with cell growth on the collagen started earlier within the device itself and then on the bed of the patient's wound, being always supported by the collagen sheet, results in a decisive shortening of the times during which the wound remains exposed with danger of contamination.

Since the cells are cultured on the collagen layer, there are no problems of fragility of the flap of epithelial tissue cultured in the laboratory and then applied occurring with the prior art.

Compared to transplantation from autologous skin, the amount of skin to be taken in the case of the device of the present invention is much lower.

The method for preparing the disposable medical device defined above comprises, according to the present invention, crushing, shredding and homogenizing a flap of intact skin of the patient until the formation of a cell suspension which can be introduced into the inner chamber or tray of the external envelope, then sealed and sterilized, to adhere to the collagen sheet.

### DESCRIPTION OF THE DRAWINGS

The constructional features of the device according to the invention will become apparent from the following detailed description of a possible embodiment thereof, shown by way of a non-limiting example in the accompanying drawings, in which:

FIG. 1 shows a top plan view of an example of the device according to the invention with an external envelope sectioned according to the line I-I in FIG. 2;

FIG. 2 shows a sectional view of the same device according to line II-II in FIG. 1;

FIG. 3 shows a plan view of a modified embodiment of the device shown in the preceding figures.

### DETAILED DESCRIPTION OF THE INVENTION

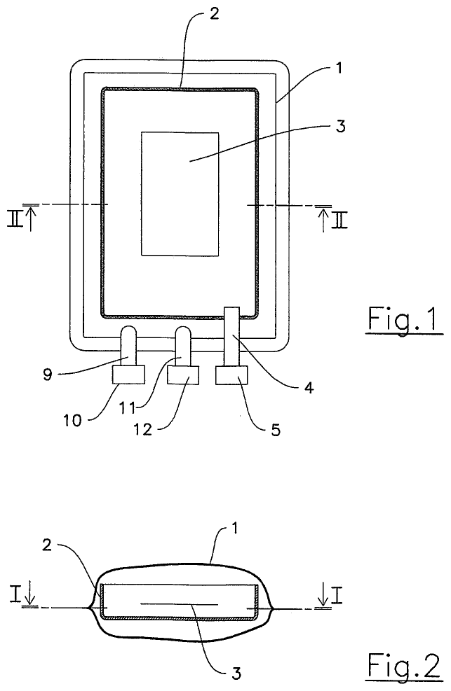

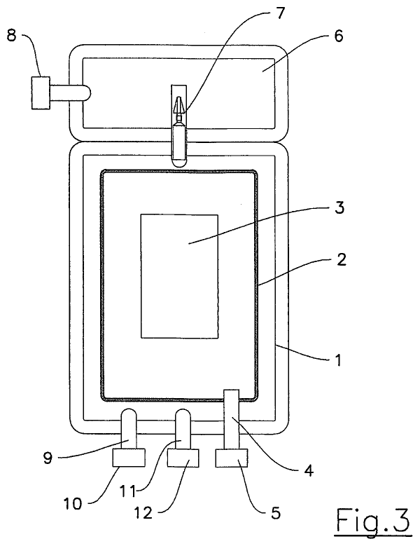

The device diagrammatically shown in FIG. 1 and FIG. 2 comprises a sealed and sterilized external envelope 1, in the shape of a three-dimensional bag, inside which a tray or cup 2 made of plastic or glass is arranged, where a collagen sheet 3 is accommodated. The tray 2 is also intended to contain, in addition to the collagen sheet 3, a homogenate of skin cells and a culture medium.

The sterile envelope 1 is made of biocompatible plastic material such as EVA (ethylene-vinyl acetate) or PVC (polyvinyl chloride).

The tray 2 is made of biocompatible plastic materials such as PET-G (polyethylene terephthalate copolyester), PP (polypropylene), PE (polyethylene), polystyrene, nylon or glass.

The structure used as a substrate for the growth of epithelial cells, i.e. the collagen sheet 3, is a native type I collagen in the form of a spongy sheet or film obtained from bovine or equine flexor tendons with one of the known techniques such as that described in U.S. Pat. No. 5,783,983. Collagen sterilization is achieved by gamma or beta irradiation. Collagen can be alone or added with glycosaminoglycans (GAG), preferably chondroitin sulfate.

From the tray 2 and through the corresponding lower part of the envelope or bag 1 a flexible tube 4 extends outwards, which ends with a sealed connector 5, through which it is possible to introduce a homogenized suspension of autologous epithelial cells into the chamber or cup 2, intended to adhere and grow on the surface of the collagen structure 3 to form a device which is ready for transplantation onto the patient's wound bed.

The connector 5 is of a type which can be pierced with a syringe needle or luer and capable of closing and self-sealing after extraction of the needle or luer at the end of the operation of introducing the cells into the chamber or tray 2. For example, but not necessarily, the connector 5 can be of the type described in EP 2 667 839 B1.

The cell homogenate introduced through the connector 5 consists of dermal-epithelial cells taken from an area of intact skin of the patient, more precisely from the epidermis layer with possible involvement of the dermis, and subjected to shredding and homogenization by means of systems known per se. The cell homogenate can be suspended in saline to facilitate the manipulation and subsequent syringe injection on the collagen sheet 3 through the connector 5 and the tube 4.

A saline may also be present inside the chamber or tray 2, in which the culture medium will be added, for example taken from a leg 6 of the bag 1, to which it is connected by means of a fracture cone 7 and an introduction connector 8 (FIG. 3).

Next to the opening from which the tube 4 emerges there is another opening of the envelope 1 from which a tube 9 extends with a breakable closure cone (not shown), which ends with a sterile filter 10 (permeable to gas and water vapor, but impermeable to liquids) which allows the circulation of an atmosphere which is suitable for cell growth.

A further tube 11 with closure cap 12 extends from a further opening of the envelope 1 to allow the discharge of the exhausted culture medium after a time which can be evaluated over 48 hours.

It is worth noting that the present invention allows using autologous cells under growing in much shorter times (46-96 hours) than currently possible in the prior art, where epithelial tissue growths with a consistent surface which is usable for reimplantation in the patient are normally obtained within 2-4 weeks.

The basic concept of the present invention is not to expand the initial tissue, but to seed the cells of the initial crushed, shredded and homogenized explant on a collagen surface where the cell growth continues to confluence, i.e. until the cells, while growing, touch and no longer find empty spaces, partially on the culture medium for 48-96 hours and partially, until the end, directly on the wound bed.

Therefore, the present invention aims at continuing the proliferation and expansion of the dermal-epithelial cells obtained from the removal of the patient's skin (which can be only epidermis using a dermatome or epidermis and dermis using a scalpel) directly on the bed of the lesion after a short period (48-96 hours) of seeding and proliferation of the cells on the collagen substrate by means of specific culture media.

The procedure of using the device according to the present invention is as follows:
a small aliquot of skin, of about 2 cm², is shredded and homogenized using one of the known techniques and suspended in a small amount of a sterile saline;
the cell suspension is distributed on the surface of the collagen sheet 3 of the device through one of the openings of the sealed envelope 1, preferably by using a syringe without needle;
a selected culture medium, which is specific for growing fibroblasts and/or keratinocytes, is loaded inside the sealed envelope;
a bacteriostatic and bactericide solution may be added if the operator is not sure on maintaining the sterile conditions during the collection, extraction, homogenization, suspension and injection procedure;
the device is placed inside an incubator for 48 to 96 hours; if the incubation is extended to 96 hours, the culture medium is changed with a fresh one;
at the end of the incubation, the sealed envelope is opened and the collagen structure, with the surface thereof invaded by autologous skin cells under growing, is washed with sterile saline so as to be ready for the implantation on the bed of the patient's wound.

1. A disposable medical device for medication of patient's skin lesions, comprising a sealed and sterilized external envelope, inside which a chamber or tray is located, a collagen sheet is accommodated in said chamber or tray (2), on which a suspension of epithelial or dermal-epithelial cells, consisting of patient's intact skin shreds, subjected to crushing, shredding and homogenization, is adhered.

2. The disposable medical device according to claim 1, wherein from said chamber or tray and through a corresponding part of the external envelope, a flexible tube extends outwards, ending with an openable and hermetically re-closable sealed connector, which is usable to introduce said suspension of epithelial or dermal-epithelial cells into the chamber or tray.

3. The disposable medical device according to claim 2, wherein the external envelope is provided with an opening from which a further flexible tube provided with a sterile filter, permeable to gas and water vapor but impermeable to liquids, extends outwards, which further flexible tube allows the circulation of an atmosphere which is suitable for growing the cells on said collagen sheet.

4. The disposable medical device according to claim 3, wherein the external envelope is provided with a further opening from which a further tube with a closure cap extends outwards for discharging the exhausted culture medium.

5. The disposable medical device according to claim 1, wherein said external envelope includes a separable leg for containing a cell culture medium.

6. The disposable medical device according to claim 5, wherein said leg is provided with an introduction connector and communicates with the interior of the external envelope by means of a tube provided with a fracture cone.

7. A method for preparing a disposable medical device according to claim 1, wherein it includes crushing, shredding and homogenizing the cells of a flap of intact skin of the patient until the formation of a suspension of cells which can be introduced into said internal chamber or tray for adhering to said collagen sheet.

8. The method according to claim 7, wherein said suspension comprises a saline.

9. The method according to claim 7, wherein a medium for culturing the introduced cells is also introduced into said internal chamber or tray.

10. The method according to claim 7, wherein a saline is also introduced into said internal chamber or tray.

11. The method for preparing and using the disposable medical device according to claim 1, which comprises the following steps:
a small aliquot of intact skin of the patient, of 2 cm², is crushed, shredded and homogenized using one of the known techniques and suspended in a small amount of a sterile saline;
the cell suspension is distributed on the surface of the collagen sheet of the device through one of the openings of the external envelope by using a syringe without needle;
a selected culture medium, which is specific for growing fibroblasts and/or keratinocytes, is loaded inside the external envelope;
the device is placed inside an incubator for 48 to 96 hours; if the incubation is extended to 96 hours, the culture medium is changed with a fresh one;
at the end of the incubation, the external envelope is opened and the collagen sheet, with the surface thereof invaded by autologous growing skin cells is washed with sterile saline so as to be ready for the implantation on the bed of the patient's wound.

12. The method according to claim 11, wherein
a bacteriostatic and bactericide solution may be added if the operator is not sure on maintaining the sterile conditions during the collection, extraction, homogenization, suspension and injection procedure.
